SEQUENCE LISTING (10 sequences)

SEQ ID NO: 1 - DNA, 20 bp, Artificial Sequence
  Adaptor oligonucleotide
acacgaccgtcttgcctact

SEQ ID NO: 2 - DNA, 19 bp, Artificial Sequence
  Adaptor oligonucleotide
gtaggcaagacgacagctc

SEQ ID NO: 3 - DNA, 45 bp, Artificial Sequence
  Oligonucleotide primer
ggattctaatacgactcactatagggacgtgtgctcttccgatct

SEQ ID NO: 4 - DNA, 20 bp, Artificial Sequence
  Oligonucleotide primer
acacgacgctcttccgatct

SEQ ID NO: 5 - DNA, 45 bp, Artificial Sequence
  Oligonucleotide primer
ggattctaatacgactcactatagggagctgtcgtcttgcctact

SEQ ID NO: 6 - DNA, 20 bp, Artificial Sequence
  Ligated adaptor sequence in Figure 3
agtaggcaagacgacagctc

SEQ ID NO: 7 - DNA, 20 bp, Artificial Sequence
  Ligated adaptor sequence after the 1st PCR cycle in Figure 3
agtaggcaagacggtcgtgt

SEQ ID NO: 8 - DNA, 45 bp, Artificial Sequence
  Adaptor sequence after the 2nd PCR cycle in Figure 3
agtaggcaagacgacagctccctatagtgagtcgtattagaatcc

SEQ ID NO: 9 - RNA, 22 nt, Artificial Sequence
  5′ end of a RNA probe in Figure 3
gggagcugucgucuugccuacu

SEQ ID NO: 10 - RNA, 20 nt, Artificial Sequence
  3′ end of a RNA probe in Figure 3
aguaggcaagacggucgugu

(19) United States
(12) Patent Application Publication
(10) Pub. No.: US 2020/0370093 A1
(43) Pub. Date: Nov. 26, 2020

(54) Methods for preparing RNA probes for exome sequencing and for depleting organelle DNA

(71) Applicant: Invitromics Pty Ltd, Vale Park (AU)

(72) Inventor: Nader Aryamanesh, Vale Park (AU)

(21) Appl. No.: 16/758,873

(22) PCT Filed: Oct. 23, 2018
(86) PCT No.: PCT/FI2018/050774

§ 371 Date: Apr. 24, 2020

(30) Foreign Application Priority Data
Oct. 24, 2017 (FI) 20175941

(51) Int. Cl. C12Q 1/6806; C12N 15/10

(52) U.S. Cl. CPC C12Q 1/6806; C12N 15/1096

(57) ABSTRACT

The present invention provides a method for preparing RNA probes useful for exome sequencing protocols or alternatively a method for the preparation of RNA probes which can be used for the separation of circular such as organelle DNA from nuclear genome.

### FIELD OF THE INVENTION

The present invention relates to the field of DNA sequencing, particularly to next generation sequencing (NGS) and exome sequencing. The present invention provides a method for preparing RNA probes useful for exome sequencing and/or exome-bisulfite sequencing protocols or alternatively a method for the preparation of RNA probes which can be used for the separation of organelle DNA from nuclear genome.

### BACKGROUND OF THE INVENTION

Exome sequencing is one of the widely used next generation sequencing methods especially where high read depth is required for data analysis. This technology requires designing special probes for targeting exomes. Most available exome probes are offered for human and can be custom designed for some species with a reference genome; but the cost of designing and producing exome-capture probes is quite high and therefore not financially feasible. However, for species without a reference genome, exome sequencing is not possible.

Exome sequencing has been defined as sequencing of all exons of protein coding genes in the genome. Exomes covers between 1 and 2% of the genome, depending on the species. The whole-exome sequencing procedure usually includes i) fragmenting DNA samples, ii) hybridizing them with biotinylated oligonucleotide probes (baits), iii) binding the biotinylated probes to magnetic streptavidin coated beads, iv) washing away non-targeted portion of the genome, v) enriching the target samples using polymerase chain reaction (PCR) and vi) sequencing the samples and bioinformatics (Warr et al. 2015). Most of current commercially available exome capture probes/kits have been developed for human genome with limited support for non-human organisms with a reference genome (reviewed in Warr et al. 2015).

About a decade ago, base-resolution whole-genome bisulfite sequencing was developed to profile DNA methylation levels (Lister et al. 2008). Currently, it is the most powerful and mostly practiced approach for methylation profiling. However, it is less powerful approach for surveying a larger number of samples owing to the added costs of sequencing entire genomes associated with it (Urich et al. 2015). Similar to exome sequencing, researchers are looking for targeted bisulfite sequencing (exome-bisulfite sequencing) to be able to examine significant numbers of individuals with high quantitative accuracy. Currently, targeted bisulfite sequencing kit is only available for human including SeqCap Epi Choice Enrichment Kit (Roche) and TruSeq DNA Methylation Kit (IIlumina) targeting mainly the exomes of human genome. These commercial kits are designed in a way to capture the libraries after bisulfite treatment and require multiple probes for a single target, therefore, the cost of designing probes for targeted bisulfite sequencing is even higher than exome sequencing. Therefore, the exome-bisulfite sequencing in non-human species is yet to be developed because of cost involved in probe design.

Further, whole genome sequencing and whole genome bisulfite sequencing are widely used technologies in life sciences worldwide. Currently, these two technologies use whole extracted DNA from an organism for sequencing purposes. Considering that there is multiple copies of organelle genomes (mitochondria and chloroplast in plants) within a single cell compared to two copies of nuclear genome, the chance of obtaining read depth for these two organelles are subsequently much higher than read depth of nuclear DNA (Lutz et al. 2011). There are around 400 mitochondria in an Arabidopsis root cell (Kato et al. 2008). In Arabidopsis and sugar beet the chloroplast DNA copy number remains at around 1700 chloroplast DNA copies per nuclear genome. Tobacco and pea leaves have around 100 chloroplasts and up to 10 000 chloroplast DNA copies (Shaver et al. 2006). The ratio of chloroplast DNA to genomic nuclear DNA remains constant even as the ploidy level of the cell changes (Zoschke et al. 2007; Rauwolf et al. 2010). Therefore, organelle genome contamination in any species will directly affect the amount of nuclear genome being sequenced. For example, IIlumina short read sequencing of several Arabidopsis ecotypes after DNA isolation with the DNeasy Plant Maxi Kit (Qiagen) resulted in 17.7% of the aligned reads being organelle genomes (Ossowski et al. 2008). In general, researchers aim to keep organelle genome contamination below 10% to maximize nuclear genome per sequencing dollar spent (Lutz et al. 2011).

CN104962552 relates to a kit for capturing a whole-genome exon sequence. An exon capture probe is designed, and a large quantity of exomes is obtained by hybridization to perform high-throughput sequencing and human whole genome comparison. However, the probes obtained are DNA based and target only one strand of the target exomes. The efficiency of DNA probes is lower than in RNA probes. RNA probes do not hybridize to another RNA probes while DNA probes can be hybridized to other DNA probes bringing the efficiency much lower.

This invention offers new methodology for creating exome-capturing probes for all species with or without a reference genome. Therefore, no prior knowledge of the genome sequence or annotation is required. These probes can capture whole exomes including previously unknown exomes and they are more focused on the expressed genes of the genome, which are biologically important. These exome-capturing probes could be used for both exome sequencing and/or exome-bisulfite sequencing as they target both sense and antisense strands of the target DNA.

This invention also offers a new methodology for isolation of organelle genomes for organelle genome sequencing purposes as well as producing organelle-genome depletion probes to be used for depletion of organelle genome(s) from whole genome sequencing or whole genome bisulfite sequencing. These organelle-genome depletion probes could also be used for any other next generation sequencing library preparations for which removal of organelle genome is desirable to obtain pure and high reads depth.

### BRIEF DESCRIPTION OF THE DRAWINGS

FIG. 1. Flowchart showing the method steps of a preferred embodiment for whole exome RNA probe preparation.

FIG. 2. Flowchart showing the method steps of a preferred embodiment for organelle genome depletion probe preparation and the method steps of a preferred embodiment for organelle genome depletion from genomic DNA sequencing libraries.

FIG. 3. Flowchart showing a preferred detailed method in adaptor and PCR primer design to capture both sense and antisense strands of a DNA sequence.

FIG. 4. Mapping efficiency of the whole exome sequencing data for Arabidopsis thaliana, Arabidopsis lyrata and Scots pine when mapped to the reference genome or reference transcriptome.

### DETAILED DESCRIPTION OF THE INVENTION

The present invention is directed to a method for preparing RNA probes for exome sequencing and/or exome-bisulfite sequencing, the method comprising the steps of:

a) extracting and isolating total RNA from a cell or tissue sample of a eukaryote of interest or providing a ready-made total eukaryotic RNA sample; said eukaryote of interest being, e.g., an animal, plant, insect or fungal species.

b) separating mRNA from the total RNA to obtain a portion of enriched population of mRNA molecules and a portion of non-protein coding RNA; preferably an aliquot of said portion of enriched population of mRNA molecules obtained in step b) is used for normalization in step d).

c) preparing a first adaptor ligated cDNA library from said portion of enriched population of mRNA molecules, wherein said first library is preferably prepared by ligating a double-stranded adaptor sequence on both ends of a double-stranded cDNA fragment produced by cDNA synthesis;

d) preparing a second adaptor ligated cDNA library from said portion of non-protein coding RNA, wherein said second library is preferably prepared by ligating a double-stranded adaptor sequence on both ends of a double-stranded cDNA fragment produced by cDNA synthesis;

e) performing PCR enrichment of said second adaptor ligated cDNA library with a first primer pair comprising an RNA polymerase promoter sequence, preferably SP6, T3 or T7 phage RNA polymerase promoter sequence, wherein said first primer pair also comprises a sequence specific to the adaptor sequence present in the second adaptor ligated cDNA library;

f) synthesizing a first set of RNA probes by using an RNA polymerase, preferably SP6, T3 or T7 phage RNA polymerase, in the presence of the enriched cDNA library obtained from step e), wherein said RNA probes are synthesized with a selectable label, preferably with a selectable affinity label such as biotin or a derivative thereof;

g) hybridizing said first set of RNA probes with said first adaptor ligated cDNA library, separating the hybridized and non-hybridized sample and collecting the non-hybridized sample to produce a depleted-mRNA-library;

h) performing PCR enrichment of said depleted-mRNA-library with a second primer pair comprising the RNA polymerase promoter sequence, wherein said second primer pair also comprises a sequence specific to the adaptor sequence present in the first adaptor ligated cDNA library;

i) synthesizing a set of RNA probes suitable for exome sequencing by using an RNA polymerase in the presence of the enriched depleted-mRNA-library obtained from step h), wherein said RNA probes are synthesized with a selectable label, preferably with a selectable affinity label such as biotin or a derivative thereof.

In a preferred embodiment, in step c) a duplex-specific nuclease (DSN) is used after adaptor ligation or in step h) after PCR enrichment to normalize the cDNA library obtained. DSN is an enzyme that selectively cleaves dsDNA and DNA in DNA-RNA hybrid duplexes. DSN is also able to discriminate between perfectly and non-perfectly matched short duplexes. DSN is inactive towards ssDNA and RNA.

In a preferred embodiment, the above method comprises a further step of capturing exome sequences from a DNA library by contacting the set of RNA probes obtained in step i) with said library and selecting those sequences from said library which are bound to any of said RNA probes.

In another preferred embodiment, the above method comprises a further step of sequencing the sequences bound to any of said RNA probes, preferably performed as bisulfite sequencing.

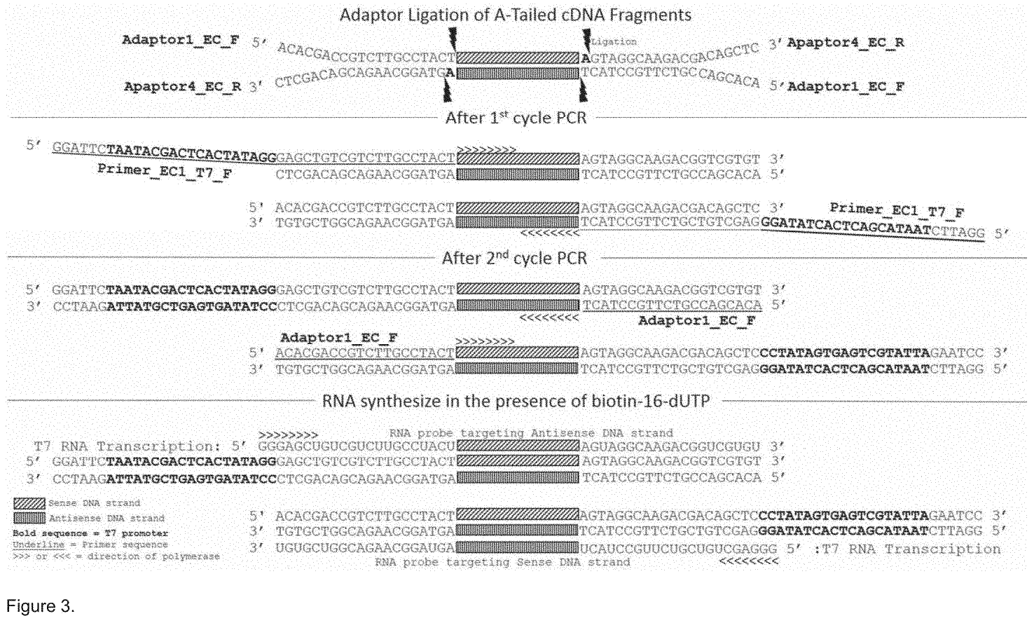

In the PCR enrichment steps of the present method, the primer pair preferably comprises a first primer having a 3′ end specific to the adaptor sequence used in the cDNA library preparation and a 5′ tail comprising said RNA polymerase promoter sequence while the second primer comprises a sequence which is specific to said adaptor sequence (see FIG. 3). Preferably, the PCR enrichment step is carried out so that the first primer having said 5′ tail is elongated in the first cycle(s) of the process and the second primer is elongated in the subsequent cycle(s) of the process. Finally, the said RNA polymerase promoter sequence is incorporated to both sense and antisense strands of original cDNA library sequences.

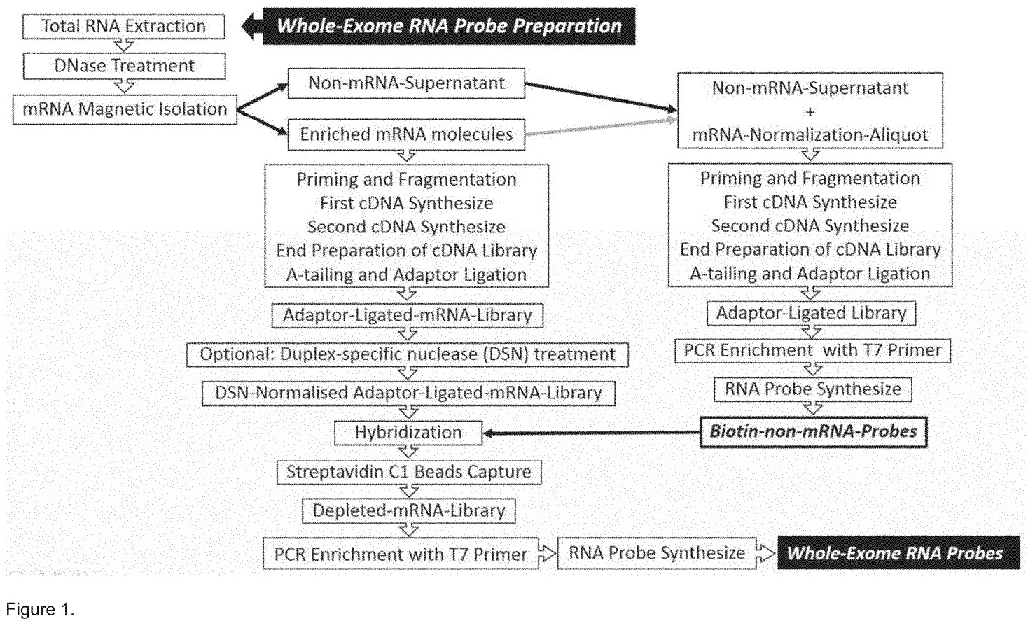

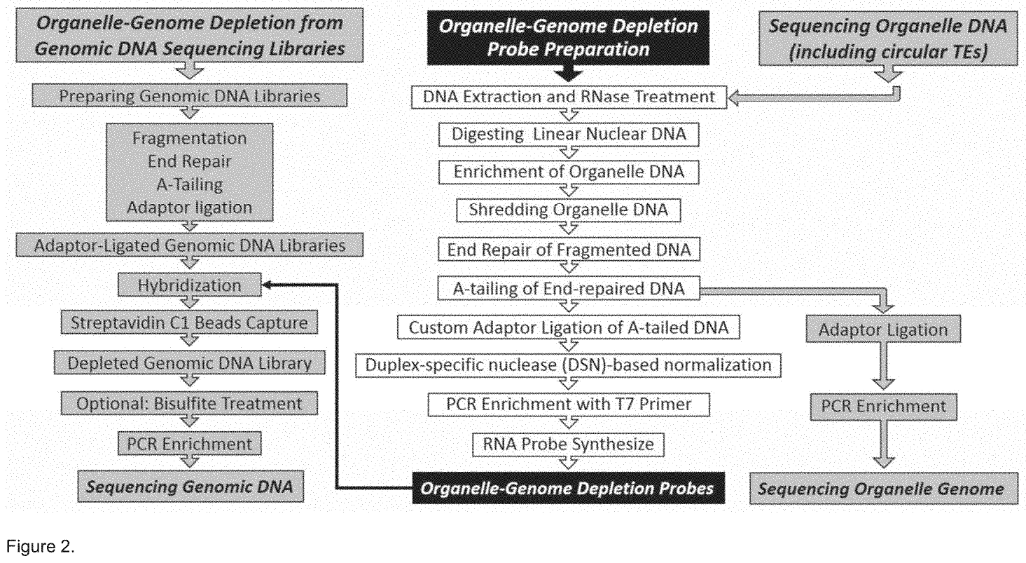

In the embodiments of the invention, the steps c) and d) preferably comprise the steps of i) priming and fragmentation of the RNA molecules, ii) first strand cDNA synthesis, iii) second strand cDNA synthesis, iv) end preparation, v) A-tailing and vi) adaptor ligation (see also FIGS. 1 and 2). An example of the preparation of an adaptor-ligated cDNA library is disclosed in Chenchik et al., 1996.

More preferably, the method of the present invention may comprise the following steps (see also FIG. 1: “Whole-Exome RNA Probe Preparation”):
[01] Total RNA extraction from animal, plant, or insect tissues (basically from any eukaryotic species) with a total RNA extraction kit (e.g. QIAGEN “RNeasy Plant Mini Kit”).
[02] DNase treatment of extracted RNA to remove genomic DNA contamination (e.g. QIAGEN “RNase-Free DNase Set” kit), followed by RNA cleanup and PCR confirmation of genomic DNA removal.
[03] mRNA isolation, fragmentation and priming of total RNA. There are variety of kits for this purpose. For instance, NEBNext Poly(A) mRNA Magnetic Isolation Module (NEB #E7490) within NEBNext® Ultra™ RNA Library Prep Kit for IIlumina (NEB #E7530) can be used.
[04] Collection of supernatant. Preferably by following the manufacturer's instruction for the NEBNext Poly(A) mRNA Magnetic Isolation Module (NEB #E7490) (except at step 16 in section 1.2 of kit producer's protocol) the supernatant is collected and kept. This collected supernatant containing non-mRNA molecules including ribosomal RNA, small RNA, and non-protein coding RNA can be called as “non-mRNA-supernatant”. Note: If another kit is used for mRNA enrichment, make sure that non-mRNA portion is also collected.
[05] The manufacturer's instructions can be followed to dilute mRNA in 17 μl of the First Strand Synthesis Reaction Buffer and Random Primer mix (2×) can be prepared following the section 1.1 of NEBNext® Ultra™ RNA Library Prep Kit for Illumina (NEB #E7530). 1/100 of mRNA is aliquoted and named “mRNA-normalization-aliquot” and kept at −20° C. freezer. The following steps are performed on the rest of the mRNA using the manufacturer's instructions: mRNA fragmentation at 94° C. for 15 minutes, First Strand cDNA Synthesis (section 1.3), Second Strand cDNA Synthesis (section 1.4), Purifying the Double-stranded cDNA (section 1.5), and End Prep of cDNA Library (section 1.6). Note: ProtoScript® II Reverse Transcriptase (M0368), RNase Inhibitor, Murine (M0314), Random Primer Mix (S1330), NEBNext® mRNA Second Strand Synthesis Module (E6111) and NEBNext® End Repair Module (E6050) could also be purchased separately and be used for these steps.
[06] Adaptor Ligation can be performed according to the manufacturer's instructions with an exception of using the “Custom-Adaptor-EC” with primers (Adaptor1_EC_F: 5′ ACA CGA CCG TCT TGC CTA CT, SEQ ID NO:1 and Apaptor4_EC_R: 5′ GTA GGC AAG ACG ACA GCT C, SEQ ID NO:2) instead of using Diluted NEBNext Adaptor. Note: There is no need to use USER® (Uracil-Specific Excision Reagent) Enzyme in this section.
[07] The Ligation Reaction can be purified using AMPure XP Beads by Beckman Coulter (section 1.8), and named “Adaptor-ligated-mRNA-library” and stored at −20° C.
[08] The “non-mRNA-supernatant” from clause [04] can be cleaned using 1.8× Agencourt AMPure XP Beads. 17 μl of the First Strand Synthesis Reaction Buffer and Random Primer mix (2×) can be prepared and in Section 1.1 of NEBNext® Ultra™ RNA Library Prep Kit for Illumina (NEB #E7530) can be added to the beads to elute non-mRNA-supernatant. The “mRNA-normalization-aliquot” from clause [05] can be added to the cleaned non-mRNA-supernatant and incubated at 94° C. for 15 minutes to fragment the RNA. The manufacturer's instructions can be followed to perform First Strand cDNA Synthesis (section 1.3), Second Strand cDNA Synthesis (section 1.4), purify the Double-stranded cDNA (section 1.5), End Prep of cDNA Library (section 1.6), Perform Adaptor Ligation using NEBNext adaptors (section 1.7) and purify the Ligation Reaction using AMPure XP Beads (section 1.8).
[09] PCR Enrichment of Adaptor Ligated DNA can be done using “Primer_T7_Fi7:

(SEQ ID NO: 3)
5' GG ATT CTA ATA CGA CTC ACT ATA GGG ACG TGT GCT
CTT CCG ATC T”,

“Primer_R _i5: 5′ A CAC GAC GCT CTT CCG ATC T” (SEQ ID NO:4) and NEBNext Q5 Hot Start HiFi PCR Master Mix (2×) by New England Biolabs (NEB #M0543). Thermocycler conditions are as follow: Initial Denaturation at 98° C. for 30 seconds, 30 cycles of Denaturation at 98° C. for 10 seconds and Annealing/Extension at 65° C. for 75 seconds, followed by 1 cycle of Final Extension at 65° C. for 5 minutes.
[10] PCR product from clause is [09] purified using, e.g., AMPure XP Beads (0.9× bead to sample ratio)
[11] RNA probe synthesis of purified PCR product from clause [10] can be performed using HiScribe™ T7 High Yield RNA Synthesis Kit (NEB # 2040) as manufacturer's instructions using modified dNTP concentration protocol including biotin-16-dUTP (Jena Bioscience #NU-803-B1016-S). After DNase I treatment, the RNA can be purified using GeneJET PCR Purification Kit (Thermo Fisher Scientific #K0701), diluted to 500 ng/μl, added 1 μl of SUPERase-In and stored at −80° C. The labeled RNA from this clause is called as “Biotin-non-mRNA-Probe”.
[12] Hybridization of “Adaptor-ligated-mRNA-library” from clause [07] with “Biotin-non-mRNA-Probe” from clause [11]. In detail, 18 μl of “Adaptor-ligated-mRNA-library” can be incubated at 95° C. for 5 min followed by 65° C. for 5 min (so called “Block A”). 1 μl of 500 ng/μl “Biotin-non-mRNA-Probe” from clause [11] is added to 1 μl of SUPERase-In and 20 μl of 2× hybridization buffer (10×SSPE, 10 mM 0.5 M EDTA, pH 8.0, 10× Denhardt's Solution, 0.2% Sodium Dodecyl Sulfate (SDS)) and the sample is incubated at 65° C. for 2 min (so called “Block B”). “Block A” and “Block B” are mixed together (total volume of 40 μl) and incubated at 65° C. overnight.

[13] Hybridized fragments from clause [12] are depleted using, e.g., Dynabeads® MyOne™ Streptavidin C1 beads. In detail, 20 μl of the Streptavidin C1 beads are washed three times with 500 μl of 1× wash buffer (5 mM Tris HCl pH 8, 0.5 mM EDTA, 1M NaCl, 0.05% tween) and after removing the wash buffer, 40 μl of 2× binding buffer (10 mM Tris HCl pH 8, 1 mM EDTA, 2 M NaCl) is added to the beads. Then, 40 μl of hybridized fragments from clause [12] is added to the beads and incubated for 30 min with rotation at room temperature. Beads are separated using magnetic rack and the supernatant is collected (throw away the beads). The supernatant is washed using AMPure XP Beads (1.6× bead to sample ratio) and eluted in 18 μl of 10 mM Tris-CI, 0.05% TWEEN®-20 solution (pH 8.0-8.5). The sample is incubated at 37° C. for 30 minutes in the presence of 1 mg/ml RNase A to remove excess RNA probes (if needed). The sample is named “depleted-mRNA-library”.

[14] PCR Enrichment of “depleted-mRNA-library” from clause [13]. PCR is performed using, e.g., NEBNext Q5 Hot Start HiFi PCR Master Mix 2× with Primer EC1 T7 F:

(SEQ ID NO: 5)
5′ GG ATT CTA ATA CGA CTC ACT ATA GGG AGC TGT CGT
CTT GCC TAC T

and Adaptorl_EC_F: A CAC GAC CGT CTT GCC TAC T (SEQ ID NO:1) with the following thermocycler condition: Initial Denaturation at 98° C. for 30 seconds, 30 cycles of Denaturation at 98° C. for 10 seconds, Annealing/Extension at 65° C. for 75 seconds, followed by 1 cycle of Final Extension at 65° C. for 5 minutes.
[15] Purification. The PCR product from clause can be [14] purified using, e.g., AMPure XP Beads (0.9× bead to sample ratio)
[16] RNA probe synthesis for cleaned PCR product from clause [15] can be performed using HiScribe™ T7 High Yield RNA Synthesis Kit (NEB #2040) as manufacturer's instructions. Modified dNTP concentration protocol including biotin-16-dUTP (Jena Bioscience #NU-803-B1016-S) can be used. After DNase I treatment, the RNA can be purified using GeneJET PCR Purification Kit (Thermo Fisher Scientific #K0701), diluted to 500 ng/μl, added 1 μl of SUPERase-In and stored at −80° C. The labeled RNA from this clause is called “Whole-Exome-Probe”.
[17] “Whole-Exome-Probe” from clause [16] can be used as capturing probes for exome library preparation and targeted-bisulfite (exome-bisulfite) library preparation.

The present invention is also directed to a method for preparing RNA capturing probes for the separation of circular DNA such as organelle DNA from nuclear genome, preferably said organelle DNA being from chloroplast or mitochondrion, the method comprising the steps of:

a) extracting and isolating total DNA from a cell or tissue sample of a eukaryote of interest or providing a ready-made total eukaryotic DNA sample;

b) digesting linear nuclear DNA obtained in step a) in the presence of exonucleases, preferably Lambda Exonuclease and Exonuclease I, or separating circular DNA from the total DNA to isolate organelle DNA and other circular DNA, or providing a ready-made sample of isolated organelle DNA;

c) fragmenting the circular DNA obtained in step b);

d) performing end repairing and dA-tailing to fragments obtained in step c);

e) performing adaptor ligation to fragments obtained in step d) to produce a DNA library of circular DNA fragments (i.e. linear or non-circular fragments originating from said circular DNA) , wherein said library is preferably prepared by ligating a double-stranded adaptor sequence on both ends of an A-tailed DNA fragment;

f) performing PCR enrichment to the DNA library obtained in step e) with a primer pair comprising an RNA polymerase promoter sequence, preferably SP6, T3 or T7 phage RNA polymerase promoter sequence, wherein said primer pair is specific to the adaptor sequence present in the DNA library;

g) synthesizing a set of RNA probes by using an RNA polymerase, preferably SP6, T3 or T7 phage RNA polymerase, in the presence of the enriched DNA library obtained from step f), wherein said set of RNA probes are suitable for depletion of fragmented circular DNA from total DNA samples of said eukaryote of interest, wherein said RNA probes are synthesized with a selectable label such as biotin or a derivative thereof.

In a preferred embodiment, the method comprises a further step of capturing circular DNA from a DNA library by contacting the set of RNA probes obtained in step g) with said library and separating those sequences from said library which are bound to any of said RNA probes from those sequences which are not bound to any of said RNA probes.

In a preferred embodiment, the method comprises a further step of sequencing the sequences bound to the RNA probes or alternatively the sequences not bound to the RNA probes. In a more preferred embodiment, the adaptor ligated DNA library of organelle DNA fragments obtained in step e) could be directly sequenced after PCR enrichment with NGS compatible indexed primers.

In a preferred embodiment, in step e) a duplex-specific nuclease (DSN) is used after adaptor ligation to normalize the circular DNA library obtained.

In another preferred embodiment, in step b) the first exonuclease digests one strand of linear dsDNA (making ssDNA) while the second exonuclease digests the remaining single stranded DNA.

Preferably, the circular DNA is organelle DNA from chloroplast and/or mitochondrion or circular transposable DNA. Transposable elements (TEs) may be active in a eukaryotic cell and may produce circular DNA (and sometimes may be present even as many copies as organelle DNA).

More preferably, the method of the present invention may comprise the following steps (see also FIG. 2: “Organelle Genome Depletion Probe Preparation”):
[18] Isolation of organelle genome(s) including mitochondria and/or chloroplast (in plants). 500 ng of total extracted DNA is treated with Lambda Exonuclease (NEB #M0262) at 37° C. for 2 hours followed by Exonuclease I (NEB #M0293) digestion at 37° C. for 2 hours. These enzymes digest linear nuclear DNA while they are not able to digest supercoiled and circular mitochondria and/or chloroplast DNA. There are also other methods available for isolation of organelle genomes, which could be used if needed.
[19] Cleaning up digested DNA using, e.g., PCR purification kit such as GeneJET PCR Purification Kit (Thermo Fisher Scientific # K0701).
[20] Organelle- and nuclear-DNA specific primers are used to confirm successful removal of nuclear DNA using PCR and for enrichment of organelle DNA. If necessary, the above two clause [18] and [19] can be repeated to make sure the nuclear DNA specific primers do not amplify any fragments.
[21] Shredding cleaned organelle DNA from clause [19]. Bioruptor can be used for shredding DNA for 200-300 bp fragments using 30″/90″ (On/Off cycle time) for 30 minutes.
[22] Optional application: If the purpose of project is to sequence organelle genome(s), DNA library can be prepared from shredded DNA from clause [21] using, e.g., commercially available DNA library preparation kits (e.g. NEBNext® DNA Library Prep Master Mix Set for Illumina for library prep kit #E7370).
[23] Performing end repair of fragmented DNA from clause [21] followed by product cleanup using AMPure XP beads (1.6× beads to sample ratio). The chemicals in NEBNext® DNA Library Prep Master Mix Set for Illumina for library prep kit (NEB #E7370) can be used to perform this step.

[24] Performing dA-Tailing of End Repaired DNA from clause [23] followed by product cleanup using AMPure XP beads (1.6× beads to sample ratio). The chemicals in NEBNext® DNA Library Prep Master Mix Set for Illumina for library prep kit (NEB #E7370) can be used to perform this step.
[25] Performing Adaptor Ligation of dA-Tailed DNA from clause [24] using Adaptor1_EC_F: 5′ ACA CGA CCG TCT TGC CTA CT (SEQ ID NO:1) and Apaptor4_EC_R: 5′ GTA GGC AAG ACG ACA GCT C (SEQ ID NO:2) instead of using Diluted NEBNext Adaptor. The chemicals in NEBNext® DNA Library Prep Master Mix Set for Illumina for library prep kit (NEB #E7370) can be used to perform this step, followed by product cleanup using, e.g. AMPure XP beads (1.6× beads to sample ratio).
[26] PCR Enrichment of adaptor ligated DNA from clause [25]. PCR can be performed using NEBNext Q5 Hot Start HiFi PCR Master Mix (2×) with Primer_EC1_T7_F:

(SEQ ID NO: 5)
5′ GG ATT CTA ATA CGA CTC ACT ATA GGG AGC TGT CGT
CTT GCC TAC T

and Adaptor1_EC_F: A CAC GAC CGT CTT GCC TAC T (SEQ ID NO:1) with the following thermocycler condition; Initial Denaturation at 98° C. for 30 seconds, 30 cycles of Denaturation at 98° C. for 10 seconds and Annealing/Extension at 65° C. for 75 seconds, followed by 1 cycle of Final Extension at 65° C. for 5 minutes.

[27] The PCR product from clause [26] is purified using, e.g. AMPure XP Beads (0.9× bead to sample ratio)
[28] RNA probe for cleaned PCR product from clause [27] can be synthesized using HiScribe™ T7 High Yield RNA Synthesis Kit (NEB #2040) as manufacturer's instructions using modified dNTP concentration protocol including biotin-16-dUTP (Jena Bioscience #NU-803-B1016-S). After DNase I treatment, the RNA can be purified using GeneJET PCR Purification Kit (Thermo Fisher Scientific #K0701), diluted to 500 ng/pl, added 1 μl of SUPERase-In and stored at −80° C. The biotin labeled RNA from this clause is called “Organelle-depletion-Probe”.
[29] “Organelle-depletion-Probe” from clause [28] can be used as capturing probes for depletion of organelle genome from whole genome sequencing (re-sequencing), whole genome de novo sequencing, exome sequencing, targeted sequencing, targeted-bisulfite (exome-bisulfite) sequencing, whole genome bisulfite sequencing, reduced representation bisulfite sequencing, directional or non-directional RNA sequencing, RAD sequencing, ddRAD sequencing, genotyping by sequencing library preparations or any other available sequencing approaches.

In another preferred embodiment, the present invention also provides the following method for the depletion of organelle DNA from DNA libraries (see FIG. 2, “Organelle Genome Depletion from Genomic DNA sequencing Libraries”):

[30] Preparing the next generation sequencing libraries using. e.g., commercial kits according to manufacturer's instructions until cleaned “Adapter-ligated-library” for any sequencing platform including whole genome sequencing (re-sequencing), whole genome de novo sequencing, exome sequencing, targeted sequencing, directional or non-directional RNA sequencing, RAD sequencing, ddRAD sequencing, genotyping by sequencing library preparations or any other available sequencing approaches is ready. For targeted-bisulfite (exome-bisulfite) sequencing, whole genome bisulfite sequencing and reduced representation bisulfite sequencing, prepare the libraries until cleaned adaptor ligated DNA is ready without performing bisulfite treatment.

[31] Hybridization of Adaptor-ligated-library from clause [07] [30] with “Organelle-depletion-Probe” from clause [28]. In detail, 18 μl of Adaptor-ligated-library from clause [30] is incubated at 95° C. for 5 min followed by 65° C. for 5 min (so called “Block A”). 1 μl of 500 ng/μl “Organelle-depletion-Probe” from clause [28] is added to 1 μl of SUPERase-In and 20 μl of 2× hybridization buffer (10×SSPE, 10 mM 0.5 M EDTA, pH 8.0, 10× Denhardt's Solution, 0.2% Sodium Dodecyl Sulfate (SDS)) and incubated at 65° C. for 2 min (so called “Block B”). “Block A” and “Block B” are mixed together (total volume of 40 μl, so called “Block H”) and incubated at 65° C. overnight.

[32] Depletion of hybridized fragments from clause [31] using, e.g., Dynabeads® MyOne™ Streptavidin C1 beads. In detail, 20 μl of the Streptavidin C1 beads are washed with 500 μl of 1× wash buffer (5 mM Tris HCl pH 8, 0.5 mM EDTA, 1M NaCl, 0.05% tween), after removing the wash buffer, 40 μl of 2× binding buffer (10 mM Tris HCl pH 8, 1 mM EDTA, 2 M NaCl) is added to the beads. 40 μl of hybridized fragments (“Block H”) from clause [31] is added to the beads and incubated for 30 min with rotation at room temperature. Separate the beads in a magnetic rack and collect the supernatant (beads containing captured organelle genome fragments were thrown away). The supernatant is washed using AMPure XP Beads (1.6× bead to sample ratio) and eluted in 18 μl of 10 mM Tris-Cl, 0.05% TWEEN®-20 solution (pH 8.0-8.5). The sample is incubated at 37° C. for 30 minutes in the presence of 1 mg/ml RNase A to remove excess RNA probes (if needed). The sample is then named “depleted-library”.
[33] The rest of original library preparation protocol (left over from clause [30] can be performed on the “depleted-library” from clause [32] according to the kit's specific manufacturer's instructions and then sent for sequencing.

The present invention is also directed to a set of RNA probes obtained by the first mentioned method above, wherein said set of RNA probes is suitable for selecting exome sequences of a eukaryotic species from a cDNA library. Each of the RNA probes comprises copies of cDNA library adaptor sequences flanking a eukaryotic genomic strand and the first nucleotide at the 5′ end of the probe is G as the probe is produced by a RNA polymerase as defined above. The set of RNA probes target both sense and antisense strands of the exome sequences. Preferably, the 5′ adaptor sequence and the 3′ adaptor sequence flanking a eukaryotic genomic strand in each of the RNA probes comprise at least 8-12 contiguous complementary nucleotides (see e.g. FIG. 3).

The present invention also provides a set of RNA probes obtained by the latter method mentioned above, wherein said set is suitable for selecting circular organelle sequences of a eukaryotic species from a DNA library. Each of the RNA probes comprises copies of DNA library adaptor sequences flanking a eukaryotic organelle strand and the first nucleotide at the 5′ end of the probe is G. The set of RNA probes target both sense and antisense strands of the organelle sequences. Preferably, the 5′ adaptor sequence and the 3′ adaptor sequence flanking a eukaryotic organelle strand in each of the RNA probes comprise at least 8-12 contiguous complementary nucleotides (see e.g. FIG. 3).

The total length of each RNA probe in said sets is preferably 100-400 nt, more preferably 100-300 nt or 150-250 nt, most preferably about 200 nt. The length of said adaptor sequences in said probes is preferably 18-25 nt, more preferably 20-22 nt. Even more preferably, said adaptor sequences are not complementary to NGS adaptor sequences to prevent capturing non-specific fragments from DNA libraries comprising common NGS adaptors. Preferably, the probes comprise labelled U nucleotides and the preferred label is biotin.

In its further embodiment, the invention also provides a kit for exome probe preparation or organelle depletion probe preparation, wherein said kit comprises a first and a second adaptor oligonucleotide for cDNA or DNA library preparation, wherein said adaptor oligonucleotides are at least partly complementary to each other, and a primer pair for PCR enrichment, wherein the first primer of said primer pair has a 3′ end specific or complementary to the first adaptor oligonucleotide and a 5′ tail comprising a RNA polymerase promoter sequence and the second primer comprises a sequence which is specific or complementary to the second adaptor oligonucleotide. Preferably, said second adaptor oligonucleotide and the second primer have identical sequences. The length of the said adaptor oligonucleotides is preferably 18-25 nt, more preferably 19-20 nt or 20-22 nt. As above, said adaptor sequences should preferably not be complementary to NGS adaptor sequences. Said adaptor oligonucleotides are preferably suitable for ligation to A-tailed cDNA/DNA fragments. The adaptor oligonucleotides and PCR enrichment primers are designed so that they target both sense and antisense strands of the target cDNA or DNA library.

The present invention is further described in the following Experimental Section, which is not intended to limit the scope of the invention.

### EXPERIMENTAL SECTION

Example 1—Whole Exome Sequencing of Arabidopsis thaliana, Arabidopsis lyrata and Scots Pine

This invention has been tested in whole-exome sequencing of three different species including A. thaliana (small genome size of around 139 MB with a good quality reference genome), A. lyrata (small genome size of around 207 MB with a draft reference genome) and Scots pine (huge genome of around 20 GB with no reference genome). One sample of A. thaliana from Col ecotype, 2 samples of A. lyrata from Spiterstulen population and two samples from Scots pine (one from needle and one from megagametophyte tissues) were selected for this experiment.

Total RNA was extracted from tissues using either RNeasy Mini Kit (QIAGEN) for A. thaliana, A. lyrata or Scots pine megagametophyte tissues and Spectrum Plant Total RNA Kit (Sigma) with protocol B for Scots pine Needles. Genomic DNA was removed from the samples using “RNase-Free DNase Set” kit (QIAGEN) according to manufacturer's instructions followed by ethanol precipitation of RNA. The quality of RNA (RIN; RNA Integrity Number) was measured with Bioanalyzer using Agilent RNA 6000 Pico Kit. In this invention, NEBNext Poly(A) mRNA Magnetic Isolation Module (NEB #E7490) which is part of NEBNext® Ultra™ RNA Library Prep Kit for Illumina® (NEB #E7530) was used with the manufacturer's instructions with some modifications as follow. Note: NEBNext Poly(A) mRNA Magnetic Isolation Module (NEB #E7490) enriches majority of mRNA however around 1% of rRNA and non-mRNA molecules remains in the enriched mRNA. Therefore, the following protocol is designed to even remove the remaining 1% rRNA/non-mRNA molecules in the library and to normalize the probes.

In section 1.2, step 16 of NEB #E7530 manual, the supernatant was not thrown away and instead it was collected, labeled as “non-mRNA-supernatant” and stored at −20° C. for later use. The protocol was followed on the beads in the section 1.2, step 16 of NEB #E7530 manual. Before RNA fragmentation, an aliquot of mRNA (1/100) was collected and labeled as “mRNA-normalization-aliquot” and stored at −20° C. for later use. RNA fragmentation was performed on the main aliquot of mRNA at 94° C. for 10 minutes instead of 15 minutes to yield bigger fragments (around 300 bp). In the “First strand cDNA synthesis” step, the incubation time was increased from 15 minutes to 50 minutes as recommended for bigger fragments. The “Second Strand cDNA Synthesis” was performed according to manufacturer's instructions followed by bead purification of the double-stranded cDNA using 1.8× Agencourt AMPure XP beads and “End Prep of cDNA library”. Adaptor ligation was performed using Adaptorl_EC_F: 5′ ACA CGA CCG TCT TGC CTA CT (SEQ ID NO:1) and Apaptor4_EC_R: 5′ GTA GGC AAG ACG ACA GCT C (SEQ ID NO:2) instead of using Diluted NEBNext Adaptor without performing USER Enzyme treatment step. The adaptor-ligated libraries were purified using AMPure XP Beads and labeled as

“Adaptor-ligated-mRNA-library” and stored at −20° C.

“Non-mRNA-supernatant” was cleaned using 1.8× Agencourt AMPure XP Beads and eluted in 17 μl of the First Strand Synthesis Reaction Buffer and Random Primer mix (2×) prepared in Section 1.1 of NEBNext® Ultra™ RNA Library Prep Kit for IIlumina (NEB #E7530). “mRNA-normalization-aliquot” was added to the cleaned non-mRNA-supernatant and incubated at 94° C. for 10 minutes to fragment the RNA. The manufacturer's instructions was followed to perform First Strand cDNA Synthesis (section 1.3), Second Strand cDNA Synthesis (section 1.4), Purifying the Double-stranded cDNA (section 1.5), End Prep of cDNA Library (section 1.6), Adaptor Ligation using NEBNext adaptors (section 1.7) and Purify the Ligation Reaction Using AMPure XP Beads (section 1.8).

PCR Enrichment of Adaptor Ligated DNA was performed with “Primer_T7_Fi7:

(SEQ ID NO: 3)
5′ GG ATT CTA ATA CGA CTC ACT ATA GGG ACG TGT GCT
CTT CCG ATC T”

and “Primer_R _i5: 5′ A CAC GAC GCT CTT CCG ATC T” (SEQ ID NO:4) using NEBNext Q5 Hot Start HiFi PCR Master Mix, 2×. Thermocycler condition was as follow: Initial Denaturation at 98° C. for 30 seconds, 30 cycles of Denaturation at 98° C. for 10 seconds and Annealing/Extension at 65° C. for 75 seconds, followed by 1 cycle of Final Extension at 65° C. for 5 minutes. PCR products were cleaned using AMPure XP Beads (0.9× bead to sample ratio)

RNA probes were synthesis from cleaned PCR product using HiScribe™ T7 High Yield RNA Synthesis Kit (NEB #2040) as manufacturer's instructions using modified dNTP concentration protocol in the presence of biotin-16-dUTP (Jena Bioscience #NU-803-B1016-S). After DNase I treatment, the RNA was purified using GeneJET PCR Purification Kit (Thermo Fisher Scientific #K0701), the concentration was adjusted to 500 ng/μl and 1 μl of SUPERase-In was added to the sample and stored at −80° C. The biotin labeled RNA was named as “Biotin-non-mRNA-Probe”.

The “Adaptor-ligated-mRNA-library” was hybridized with “Biotin-non-mRNA-Probe”. In detail, 18 μl of “Adaptor-ligated-mRNA-library” was incubated at 95° C. for 5 min, then 65° C. for 5 min (so called “Block A”). 1 μl of 500 ng/pl “Biotin-non-mRNA-Probe” was added to 1 μl of SUPERase-In and 20 μl of 2× hybridization buffer (10×SSPE, 10 mM 0.5 M EDTA, pH 8.0, 10× Denhardt's Solution, 0.2% Sodium Dodecyl Sulfate (SDS)) and incubated at 65° C. for 2 min (so called “Block B”). “Block A” and “Block B” were mixed together (total volume of 40 μl, so called “Block H”) and incubated at 65° C. overnight. The hybridized samples were washed using Dynabeads® MyOne™ Streptavidin C1 beads as follow. 20 μl of the Streptavidin C1 beads washed with 500 μl of pre-heated (65° C.) 1× wash buffer (5 mM Tris HCl pH 8, 0.5 mM EDTA, 1M NaCl, 0.05% tween), after removing the wash buffer, 40 μl of pre-heated (65° C.) 2× binding buffer (10 mM Tris HCl pH 8, 1 mM EDTA, 2M NaCl) was added to the beads. 40 μl of hybridized fragments (“Block H”) was added to the beads and incubated for 30 min with rotation at 65° C. The samples were placed in a magnetic rack and the supernatant was collect. The beads containing captured non-mRNA fragments was thrown away. The supernatant was washed using AMPure XP Beads (1.6× bead to sample ratio) and eluted in 18 μl of 10 mM Tris-Cl, 0.05% TWEEN®-20 solution (pH 8.0-8.5). The sample was incubated at 37° C. for 30 minutes in the presence of 1 mg/ml RNase A to remove excess RNA probes (if needed). The sample named as “depleted-mRNA-library”.

PCR Enrichment of “depleted-mRNA-library” was performed using NEBNext Q5 Hot Start HiFi PCR Master Mix 2× with Primer_EC1_F:

(SEQ ID NO: 5)
5′ GG ATT CTA ATA CGA CTC ACT ATA GGG AGC TGT CGT
CTT GCC TAC T

and Adaptor1_EC_F: A CAC GAC CGT CTT GCC TAC T (SEQ ID NO:1) with the following thermocycler condition: Initial Denaturation at 98° C. for 30 seconds, 30 cycles of Denaturation at 98° C. for 10 seconds and Annealing/Extension at 65° C. for 75 seconds, followed by 1 cycle of Final Extension at 65° C. for 5 minutes. PCR product was cleaned using AMPure XP Beads (0.9× bead to sample ratio). RNA probe synthesis was perform using 1 pg of PCR product as template using HiScribe™ T7 High Yield RNA Synthesis Kit (NEB # 2040) as manufacturer's instructions using modified dNTP concentration protocol in the presence of biotin-16-dUTP (Jena Bioscience #NU-803-B1016-S). After DNase I treatment, the RNA was purified using GeneJET PCR Purification Kit (Thermo Fisher Scientific # K0701), diluted to 500 ng/μl, 1 μl of SUPERase-In was added to RNA and stored at −80° C. The labeled RNA named as “Whole-Exome-Probe” was used as capturing baits for exome library preparation and targeted-bisulfite (exome-bisulfite) library preparation as follow.

High molecular weight DNA was extracted from A. thaliana, A. lyrata and scots pine and RNA was removed by RNase A (incubation at 37° C. for 30 minutes). 1 μg of DNA was shredded to around 300 bp fragments using Bioruptor (30 sec/90 sec On/Off cycle time for 30 minutes). NEBNext® Ultra™ DNA Library Prep Kit for Illumina (E7370) was used for library prep of as manufacturer's instruction until “Size Selection of Adaptor Ligated DNA” step. The size selected product named as “adaptor-ligated-DNA”

The “adaptor-ligated-DNA” was hybridized with “Whole-Exome-Probe”. In detail, 2.5 μg of salmon sperm DNA (ThermoFisher Scientific #15632011) was added to 15.5 μl of “adaptor-ligated-DNA” and incubated at 95° C. for 5 min, then 65° C. for 5 min (so called “Block A”). 1 μl of 500 ng/μl “Whole-Exome-Probe” was added to 1 μl of SUPERase-In and 20 μl of 2× hybridization buffer (10× SSPE, 10 mM 0.5 M EDTA, pH 8.0, 10× Denhardt's Solution, 0.2% Sodium Dodecyl Sulfate (SDS)) and incubated at 65° C. for 2 min (so called “Block B”). “Block A” and “Block B” were mixed together (total volume of 40 μl) and incubated at 65° C. for 66 hours (Gnirke et al. 2009). The hybridized samples were washed using Dynabeads® MyOne™ Streptavidin C1 beads as follow. 20 μl of the Streptavidin C1 beads washed three times with 200 μl of binding buffer, then the beads were re-suspended in 70 μl of binding buffer and warmed to hybridization temperature (65° C.). 40 μl of hybridized fragments was added to the beads and incubated for 30 min with occasional agitation at hybridization temperature (65° C.). The samples were placed in a magnetic rack until the solution is cleared, then the supernatant was removed and the beads were washed three times with 500 μl of pre-warmed (65° C.) wash buffer. The beads were re-suspended in 40 μl of 10 mM Tris-Cl, 0.05% TWEEN (pH 8.0-8.5) and incubated at 95° C. for 5 minutes. The beads were pelleted in a magnetic rack and supernatant which contained enriched library were collected in a new tube.

The library were PCR amplified using 2X KAPA® HiFi HotStart ReadyMix (KAPA Biosystems #KK2600) and indexed using forward and reverse library primers (NEBNext #E7600). The amplified library was washed 2 times with AMPure XP Beads (0.9× bead to sample ratio) to remove primers dimers.

The quality and size of libraries were analysed by Bioanalyzer using Agilent High Sensitivity DNA Analysis Kits. The libraries were quantified using KAPA Library Quantification Kit Illumina® Platforms (KAPA Biosystems #KK4824), pooled and sequenced using Illumina platform, NextSeq500.

Results

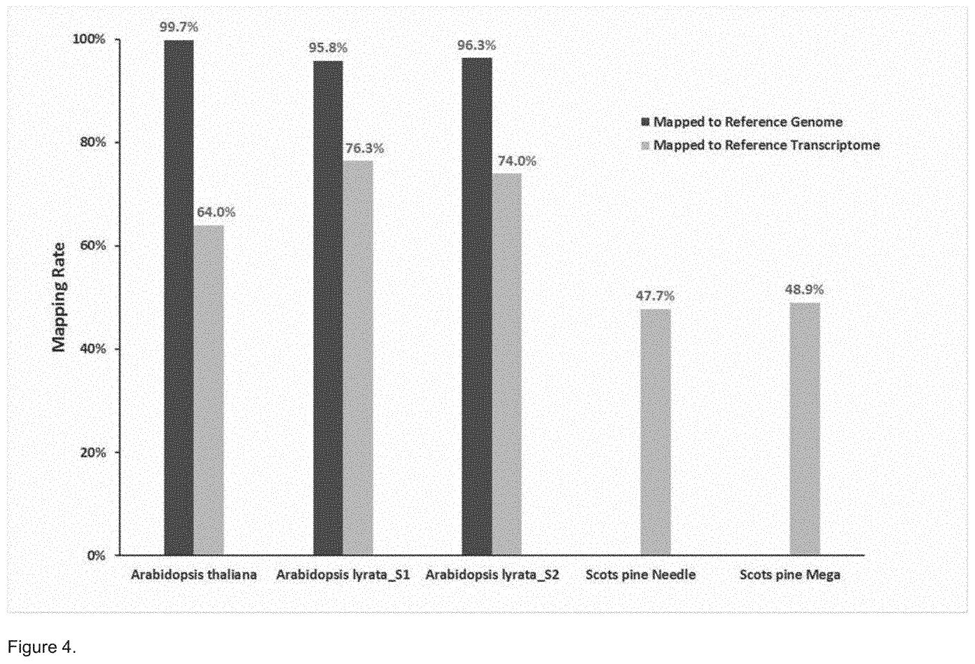

The procedure outlined in this invention was used to produce whole exome capturing probes and test the efficiency of the probes in exome sequencing of three different species including A. thaliana, A. lyrata and Scots pine. Mapping efficiency of the reads was calculated for both reference genomes (for A. thaliana and A. lyrata) and reference transcriptomes (all three species). In A. thaliana, 99.7% of the reads were mapped to the reference genome and 64% of the reads were mapped to the reference transcriptome (FIG. 4). Annotation file of A. thaliana (tair10) has 217,183 exomes and 65,255 UTRs with an exome-wide average of around 297 bp and average UTR-wide average of around 163 bp. Considering that the average fragment length for exome sequencing libraries was around 300 bp, it suggests that around 35% of the reads captured adjunct regions of the exomes and UTRs (around 150 bp of either introns or promoters). This information is very valuable when using this invention as a tool for exome sequencing and annotation of a transcriptome reference for species with no reference genome or a species with fragmented genome.

In A. lyrata, 95.8% of the reads were mapped to the A. lyrata's reference genome and 76.3% of the reads were mapped to the A. lyrata's reference transcriptome (FIG. 4). The current annotation file of A. lyrata has 170,346 exomes and 55,383 UTRs with an exome-wide average of around 222 bp and UTR-wide average of around 61 bp. Around 20% of A. lyrata's reads captured adjunct regions of the exomes and UTRs (around 150 bp of either introns or promoters).

In A. thaliana, 68.7% of exons (149,317) are larger than 100 bp which is comparable to A. lyrata with 63.2% of exons (107,734) having a minimum of 100 bp. However, the number of UTRs bigger than 100 bp was 63% and 19.8% for A. thaliana and A. lyrata, respectively. Therefore, it is expected to capture more promoter regions in A. thaliana than that in A. lyrata because the majority of UTRs in A. lyrata are smaller than 100 bp and these regions are unlikely to be pulled down in whole exome sequencing. This was due to higher mapping efficiency of A. lyrata compared to A. thaliana when they mapped to transcriptome reference.

There is no genome reference for Scots pine, however, there is a draft transcriptome reference for this genome with 36,106 coding genes (http://bioinformatics.psb.ugent.be). Whole exome sequencing was performed for Scots pine using both needle and megagametophyte tissues. For both tissues, around 48% of the reads were mapped to the transcriptome reference (FIG. 4). When compared to Arabidopsis species, current transcriptome reference for Scots pine is missing around 16-28% of the genes in the genome. Furthermore, the current transcriptome reference for Scots pine lacking information about exon-intron boundaries, making it very inefficient if traditional exome capturing probes designed for this species. This invention not only captures majority of exomes but also gives an opportunity to correct the current transcriptome reference of Scots pine for exon-intron boundaries.

In A. thaliana, this invention was able to capture exomes of around 35,340 genes (99.9%) out of total 35,386 genes with a minimum read depth of 10×. The captured portion of the exomes was 32,808,497 bp (51.1%) out of the total 64,249,826 bp with a minimum of 10× read depth.

In A. lyrata, 29,289 genes (89.7%) of total 32,667 genes were captured with this invention accounting for 60% of the exomes (23,598,131 bp out of 38,929,289 bp) a minimum read depth of 10×. The commonly captured genes between two biological samples in A. lyrata was 85.3%, demonstrating the reproducibility of whole exome capturing probes used in this invention.

In Scots pine, exome capture were performed in both needles and megagametophytes. 22,442 genes (62%) in needles and 22,676 (62%) in megagametophytes were captured out of total of 36,106 genes in known Pinus sylvestris transcriptome. This invention captured around 7,914,639 bp (26.5%) and 9,110,635 bp (30.5%) out of total 29,877,965 bp of Scots pine transcriptome in needles and megagametophytes, respectively, sharing 71° A of the captured genes using different RNA probes from different tissues.

Discussion

Exome sequencing is powerful next generation sequencing technique specially when the genome size is too large or high depth reads is essential for downstream bioinformatics. There are three platforms for exome sequencing in human including NimbleGen, Agilent and Illumina which capture between 40% and 70% of the targets (around 50-60 Mb target in human) depending the platform. Although all platforms are targeting the human exome, there is surprisingly little overlap (26.2 Mb) between the three platforms. Illumina targets more untranslated regions (UTRs) compared to NimbleGen's and Agilent's. Illumina has 22.5 Mb of unique targets (21.8 Mb of these are UTRs) while NimbleGen and Agilent have 16.1 Mb and 7 Mb of unique targets, respectively (Warr et al. 2015). These differences in target coverage makes data comparisons difficult as some targets are missing in some platforms.

If a reference genome exists for species, NimbleGen and Agilent companies support designing and providing probes. However, the process is very costly and has been offered for only limited species with efficiency being much lower than human exome capture rate. If a reference genome does not exist for species, exome library kit has not been offered at all. In some cases, close relative species has been used as a reference genome to design probes but shows high level of no-specific capture.

This invention requires no reference genome with annotation for designing the probes or downstream bioinformatics and it allowed creating a biotinylated probe from the RNA of the same species. Therefore, the probes from this invention were highly specific to the species. In species without a reference genome, some researchers use relative species to design probes and perform exome sequencing which could lower the efficiency even further down because of probe non-specificity.

Therefore, this innovation is an ideal solution for providing an opportunity for academic institutions or companies to head start with exome sequencing without waiting years for a reference genome to be published.

Targeted bisulfite sequencing can be performed either by bisulfite conversion of hybrid-selected native DNA (Lee et al. 2011) or by hybrid selection of bisulfite converted DNA (Allum et al. 2015; Li et al. 2015). Current commercially available exome capture kits only target one strand of the DNA (either sense or antisense). For targeted bisulfite sequencing, it is required to sequence both strands of DNA to investigate the methylation profile of species under certain conditions. Recently, targeted bisulfite sequencing has been offered for human (Ziller et al. 2016) which uses SeqCap Epi technology (Roche). In this technology, the probes designed to capture the regions of interest after bisulfite treatment. This procedure requires multiple copies of probe for a single target which makes probe-designing process costly.

The probes with current invention, targets both strands of a target DNA, therefore, the probes could be used for whole exome sequencing as well as whole exome bisulfite sequencing. In case of whole exome bisulfite sequencing, bisulfite conversion needs to be performed on hybrid-selected native DNA using probes from this invention. Currently, there is limited reports of targeted bisulfite sequencing for non-human species with a reference genome. As mentioned above, there are some kits available for targeted bisulfite sequencing (e.g. Roche's SeqCap Epi Choice Enrichment Kit) but native DNA capture happens after bisulfite treatment and requires multiple probes for a single target. Unless they include all possible probe combinations, the outcome might be biased towards some probes. This invention will make it feasible to work in parallel on exome sequencing and exome bisulfite sequencing of any species with or without a reference genome. Currently, there is no possibility for exome sequencing in species without a reference genome. Double digest RAD sequencing (ddRAD-Seq) is the most widely used technique for studying polymorphism in non-human species without a reference genome. The ddRAD-Seq does not target the exomes; therefore, it has less significance in term of biological meaning.

The applications in biological sciences are moving towards RNA sequencing coupled with exome sequencing and methylation profiling of genic regions to answer a biological question. This invention makes it possible to combine these three approaches in all species with or without a reference genome. This invention will revolutionize the quality and quantity of meaningful science produced worldwide and will help even in improving the existing reference genomes for human or non-human species. The followings are the list of advantages over the current applications.
Enabling exome sequencing of non-human species with or without a reference genome.
Enabling exome-bisulfite sequencing for non-human species.
Non-biased exome-bisulfite (or targeted bisulfite) sequencing for human compared to current methodology (e.g. SeqCap Epi Choice Enrichment Kit).
More focused of exomes that they are biologically important (shows gene expression) and mostly related to a biological question/cues.
There is a possibility to discover new genes which are not discovered before and which might express under rare or certain conditions. It is worth highlighting that these new genes will not be picked up with RNA sequencing as well because the sequence alignment (Tophat or Star packages) is done based on the reference genome with their known annotation.
It is very cost effective comparing the cost of designing traditional probes for human or non-human species.

Example 2—Organelle genome sequencing in Arabidopsis thaliana and Arabidopsis lyrata

Organelle genome sequencing was performed using this invention on one individual of A. thaliana ecotype Col and two individuals of A. lyrata from Spiterstulen population. Organelle genomes (both mitochondria and chloroplast) were isolated as follow: 500 ng of freshly extracted DNA was digested with Lambda Exonuclease (NEB #M0262) at 37° C. for 2 hours followed by product cleanup using GeneJET PCR Purification Kit (Thermo Fisher Scientific # K0701). The cleaned digested product was digested again with Exonuclease I (NEB #M0293) at 37° C. for 2 hours followed by second cleanup using GeneJET PCR Purification Kit (Thermo Fisher Scientific # K0701). PCR was performed using chloroplast DNA specific primers, mitochondria DNA specific primers and Nuclear DNA specific primers to confirm removal of nuclear DNA and enrichment of organelle DNA. Organelle DNA was shredded to 300 bp fragments using Bioruptor (30″/90″ On/Off cycle time for 30 minutes). This product names as “shredded_organelle_genome”.

NEBNext® DNA Library Prep Master Mix Set for Illumina for library prep kit (NEB #E7370) was used to prepare libraries for “shredded_organelle_genome” as manufacturer's instructions. The quality and size of libraries were analysed by Bioanalyzer using Agilent High Sensitivity DNA Analysis Kits. The libraries were quantified using KAPA Library Quantification Kit Illumina® Platforms (KAPA Biosystems #KK4824), pooled and sequenced using Illumina platform NextSeq500.

Results

In order to demonstrate the efficiency of enzyme based isolation and enrichment of organelle genomes for next generation sequencing projects, organelle genomes was isolated from A. thaliana and A. lyrata as described in this invention. Whole genome sequencing libraries were prepared from the isolated organelle DNA and sequenced using Illumina platform.

The average read depth for chloroplast and mitochondria of A. thaliana were around 126× and 35×, respectively. The average read depth for chloroplast and mitochondria of A. lyrata were around 603× and 86×, respectively. In A. thaliana, 68% of the nuclear genome had no reads at all while 100% chloroplast genome had reads (Table 1). Almost all (99.7%) of chloroplast genome (154,452 bp out of 154,478 bp) had minimum read depth of 50× while, for nuclear genome, it was only 0.20% for 50× read depth (Table 1). A similar pattern was observed for A. lyrata with slightly higher nuclear genome contamination. On average, 0.62% of nuclear genome had a minimum read depth of 50× compared to A. thaliana with 0.20% (Table 2). The reason for slight overestimation could be because A. lyrata genome is not a complete genome and there is chloroplast genome contamination in the reference genome.

In contrast, A. lyrata control sample (not digested with enzymes), were also sequenced. On average, 76% nuclear genome had minimum read depth of 10× in non-digested sample while it was only 10.3% in digested sample (Table 3). These experiments clearly demonstrated that organelle genome could be enriched using the enzyme digestion method described in this invention.

Discussion

There are few methodologies for organelle genome isolation which includes i) isolation of organelle tissues from cell crude followed by DNA extraction and ii) isolation of total DNA followed by CsCl density gradient centrifugation to separate nuclear DNA from organelle DNA. In both cases, time-consuming CsCl density gradient centrifugation has been adapted as part of extraction protocol. For species with small mitochondria genomes (e.g. human or mouse), plasmid miniprep kit (Quispe-Tintaya et al. 2013) or specialized kits such as mtDNA Isolation Kit (BioVision) or Mitochondria Isolation Kit (MACS) has been used. However, there is no easy way for plant/animal species with large chloroplast (above 150,000 bp) or mitochondria (above 400,000 bp) genome sizes. In this invention, combination of Lambda Exonuclease and Exonuclease I were used to eliminate linear nuclear DNA. These enzymes has been used for purification of small plasmids but never have been tried for isolation of mitochondria or chloroplast. This methodology is very fast, cheap and it could be used for any species with varying organelle genome size. Normal DNA library preparation can be performed on the purified organelle DNA for direct sequencing of these organelles. Using this invention, a high read depth were obtained for chloroplast and mitochondria in A. thaliana and A. lyrata. The average read depth for chloroplast and mitochondria of A. thaliana were around 126× and 35×, respectively. The average read depth for chloroplast and mitochondria of A. lyrata were around 603× and 86×, respectively. Since Chloroplast genome in Arabidopsis is smaller than mitochondria genome; the efficiency of this invention was much higher for chloroplast genome.

Example 3—Whole Genome Sequencing of Arabidopsis lyrata with Organelle Genome Depletion

The “shredded_organelle_genome” from A. lyrata (one individual from Spiterstulen population) was prepared as procedure outlined in Example 2. The chemicals from NEBNext® DNA Library Prep Master Mix Set for Illumina for library prep kit (NEB #E7370) was used to prepare libraries for “shredded_organelle_genome” as manufacturer's instructions with the following modifications.

“End Repair of Fragmented DNA” was performed on “shredded_organelle_genome” followed by product cleanup using AMPure XP beads (1.6× beads to sample ratio). The “dA-Tailing of End Repaired DNA” was performed following product cleanup using AMPure XP beads (1.6× beads to sample ratio). Then, the “Adaptor Ligation of dA-Tailed DNA” step was performed using Adaptor1_EC_F: 5′ ACA CGA CCG TCT TGC CTA CT (SEQ ID NO:1) and Apaptor4_EC_R: 5′ GTA GGC AAG ACG ACA GCT C (SEQ ID NO:2) instead of using NEBNext Adaptor (note: there was no need to use USER™ Enzyme Mix). The adaptor-ligated product was cleaned and size selected (300 bp) using AMPure XP beads. “PCR Enrichment of adaptor ligated DNA” was performed using NEBNext Q5 Hot Start HiFi PCR Master Mix 2× with Primer_EC1_T7_F:

(SEQ ID NO: 5)
5′ GG ATT CTA ATA CGA CTC ACT ATA GGG AGC TGT CGT
CTT GCC TAC T

and Adaptor1_EC_F: A CAC GAC CGT CTT GCC TAC T (SEQ ID NO:1) with the following thermocycler condition: Initial Denaturation at 98° C. for 30 seconds, 30 cycles of Denaturation at 98° C. for 10 seconds and Annealing/Extension at 65° C. for 75 seconds, followed by 1 cycle of Final Extension at 65° C. for 5 minutes. The PCR product was purified using AMPure XP Beads (0.9× bead to sample ratio). The product named as “T7-ligated-PCR-product”.

RNA probe synthesis was performed using 1 μl of “T7-ligated-PCR-product” using HiScribe™ T7 High Yield RNA Synthesis Kit (NEB #2040) as manufacturer's instructions using modified dNTP concentration protocol in the presence of biotin-16-dUTP (Jena Bioscience #NU-803-B1016-S). After DNase I treatment, the RNA was purified using GeneJET PCR Purification Kit (Thermo Fisher Scientific #K0701), diluted to 500 ng/μl, added 1 μl of SUPERase-In and stored at −80° C. The biotin labeled RNA was named as “Organelle-depletion-Probe”.

To prepare DNA library for whole genome resequencing of A. lyrata, the procedure as above was performed to obtain “adaptor-ligated-DNA”. 18 μl of “adaptor-ligated-DNA” was incubated at 95° C. for 5 min, then 65° C. for 5 min (so called “Block A”). 1 μl of 500 ng/μl “Organelle-depletion-Probe” was added to 1 μl of SUPERase-In and 20 μl of 2× hybridization buffer (10×SSPE, 10 mM 0.5 M EDTA, pH 8.0, 10× Denhardt's Solution, 0.2% Sodium Dodecyl Sulfate (SDS)) and incubated at 65° C. for 2 min (so called “Block B”). “Block A” and “Block B” were mixed together (total volume of 40 μl; so called “Block H”) and incubated at 65° C. overnight. 20 μl of the Streptavidin C1 beads washed with 500 μl of pre-heated (65° C.) 1× wash buffer (5 mM Tris HCl pH 8, 0.5 mM EDTA, 1 M NaCl, 0.05% tween), after removing the wash buffer, 40 μl of pre-heated (65° C.) 2× binding buffer (10 mM Tris HCl pH 8, 1 mM EDTA, 2M NaCl) was added to the beads. 40 μl of hybridized fragments (“Block C”) was added to the beads and incubated for 30 min with occasional agitation at 65° C. The beads were pelleted in a magnetic rack and the supernatant was collected (beads were throw away). The supernatant was washed using AMPure XP Beads (1.6× bead to sample ratio) and eluted in 18 μl of 10 mM Tris-Cl, 0.05% TWEEN®-20 solution (pH 8.0-8.5). The sample was incubated at 37° C. for 30 minutes in the presence of 1 mg/ml RNase A to remove excess RNA probes (optional). The sample named as “organelle-depleted-library”. PCR enrichment was performed with indexed i7 and i5 primers on the “organelle-depleted-library” using manufacturer's instructions in NEBNext® DNA Library Prep Master Mix Set for Illumina for library prep kit (NEB #E7370). The amplified library was washed 2 times with AMPure XP Beads (0.9× bead to sample ratio).

The quality and size of libraries were analysed by Bioanalyzer using Agilent High Sensitivity DNA Analysis Kits. The libraries were quantified using KAPA Library Quantification Kit Illumina® Platforms (KAPA Biosystems #KK4824), pooled and sequenced using Illumina platform NextSeq500.

Results

As shown in Table 3, in whole genome sequencing of A. lyrata, majority of reads were belonged to organelle genomes with more than 1000 read depth. Around 8% of chloroplast genome had even higher than 10,000 read depth. In order to calculate the percentage of reads wasted by organelle genomes, two whole genome sequencing A. lyrata data (both single ended reads and paired ended reads) were analyzed (Table 4). Organelle genomes accounts for only around 0.27% of the genome in A. lyrata (521 kb out of total 207 Mb) however, there are multiple copies of organelle genomes within a cell compared to two copies of nuclear genome. Therefore, in whole genome sequencing projects it is expected to obtain more reads for organelle genomes compared to nuclear genome. In this invention, we crude extracted organelle genomes and produced capturing probes. Using these capturing probes, organelle genomes were depleted from whole genome sequencing libraries. The amount of reads for organelle genomes was significantly reduced using this invention. In normal whole genome DNA libraries, organelle genomes comprised more than 30% of the total reads while it was reduced to around 5% in organelle genome depleted libraries (this invention). This invention could be further improved by using highly pure organelle genome probes and extension of hybridization time to capture and deplete more organelle genomes.

Discussion

In order to reduce the amount of organelle genomes in genome sequencing project, some time consuming custom-made DNA extraction methods have been developed which are highly specific for the species. The efficiency of reducing organelle genomes were mostly low, ranging from 14% to 76% (Lutz et al. 2011). As an example, Lutz et al. (2011) reported that 30% of whole genome sequencing reads in Genlisea aurea were belonged to organelle genomes and it reduced to 11% using modified DNA extraction method.

To date, there is no methodology or kit available to deplete the whole organelle genome from whole genome sequencing projects. There is only kits available to deplete ribosomal RNA from RNA sequencing projects such as NEBNext® rRNA Depletion Kit (Human/Mouse/Rat) and Ribo-Zero rRNA Removal Kit (Human/Mouse/Rat).

Using this invention, organelle genome specific capturing probes were produced and used to deplete organelle genome fragments from whole genome library preparations for either whole genome sequencing or whole genome bisulfite sequencing projects. Organelle genome purification could be achieved by the methodology mentioned in this invention or by any previously reported extraction methods (CsCl gradient separation method or specialized kits). The purified organelle genomes could be converted to capturing probes and used to deplete the organelle genome from nuclear genome library preparations with the methodology stated in this invention. Using this invention, the organelle genome contamination was reduced from over 30% to 5% using crude organelle genome capturing probes; however, it is possible to achieve below 1% organelle genome contamination with some optimizations (e.g. producing probes from more pure organelle genomes or elongating the hybridization time).

TABLE 1
Depletion of linear nuclear genome and enrichment of
organelle genome using enzyme digestion in Arabidopsis thaliana.

| | Total | 1x | 1x | 20x | 20x | 50x | 50x |
|---|---|---|---|---|---|---|---|
| Chromosome | length (bp) | (bp) | (%) | (bp) | (%) | (bp) | (%) |
| Chr1 | 30,427,671 | 12,983,805 | 42.7 | 146,347 | 0.5 | 40,083 | 0.1 |
| Chr2 | 19,698,289 | 8,427,777 | 42.8 | 67,144 | 0.3 | 50,943 | 0.3 |
| Chr3 | 23,459,830 | 9,972,983 | 42.5 | 86,299 | 0.4 | 46,174 | 0.2 |
| Chr4 | 18,585,056 | 7,876,518 | 42.4 | 89,080 | 0.5 | 52,149 | 0.3 |
| Chr5 | 26,975,502 | 11,589,027 | 43.0 | 75,403 | 0.3 | 46,072 | 0.2 |
| Chloroplast | 154,478 | 154,478 | 100.0 | 154,452 | 99.9 | 154,081 | 99.8 |

TABLE 2
Depletion of linear nuclear genome and enrichment of
organelle genome using enzyme digestion in Arabidopsis lyrata.

| | Total | 1x | 1x | 20x | 20x | 50x | 50x |
|---|---|---|---|---|---|---|---|
| Chromosome | length (bp) | (bp) | (%) | (bp) | (%) | (bp) | (%) |
| Chr1 | 33,132,539 | 24,777,392 | 74.8 | 774,632 | 2.3 | 173,121 | 0.5 |
| Chr2 | 19,320,864 | 13,836,239 | 71.6 | 571,959 | 3.0 | 257,858 | 1.3 |
| Chr3 | 24,464,547 | 18,856,237 | 77.1 | 711,917 | 2.9 | 277,436 | 1.1 |
| Chr4 | 23,328,337 | 17,573,620 | 75.3 | 697,339 | 3.0 | 179,037 | 0.8 |
| Chr5 | 21,221,946 | 15,717,485 | 74.1 | 575,334 | 2.7 | 158,615 | 0.7 |
| Chr6 | 25,113,588 | 18,918,072 | 75.3 | 534,074 | 2.1 | 129,722 | 0.5 |
| Chr7 | 24,649,197 | 18,519,637 | 75.1 | 574,057 | 2.3 | 147,975 | 0.6 |
| Chr8 | 22,951,293 | 16,199,729 | 70.6 | 501,876 | 2.2 | 111,577 | 0.5 |
| Mitochondria | 366,924 | 258,016 | 70.3 | 219,772 | 59.9 | 183,250 | 49.9 |
| Chloroplast | 154,478 | 132,694 | 85.9 | 114,884 | 74.4 | 105,036 | 68.0 |

TABLE 3
Genome alignment statistics for in Arabidopsis lyrata
without enzyme digestion (control).

| | Total | 1x | 1x | 20x | 20x | 50x | 50x | 1000x | 10,000x |
|---|---|---|---|---|---|---|---|---|---|
| Chromosome | length (bp) | (bp) | (%) | (bp) | (%) | (bp) | (%) | (%) | (%) |
| Chr1 | 33,132,539 | 28,124,766 | 84.9 | 22,746,157 | 68.7 | 4,986,257 | 15.0 | 0.1 | 0.0 |
| Chr2 | 19,320,864 | 15,583,447 | 80.7 | 12,491,730 | 64.7 | 2,757,938 | 14.3 | 0.9 | 0.0 |
| Chr3 | 24,464,547 | 21,074,488 | 86.1 | 17,289,705 | 70.7 | 3,958,863 | 16.2 | 0.5 | 0.0 |
| Chr4 | 23,328,337 | 19,315,590 | 82.8 | 15,574,175 | 66.8 | 3,377,781 | 14.5 | 0.2 | 0.0 |
| Chr5 | 21,221,946 | 17,710,017 | 83.5 | 14,283,704 | 67.3 | 3,187,690 | 15.0 | 0.1 | 0.0 |
| Chr6 | 25,113,588 | 21,147,110 | 84.2 | 17,213,110 | 68.5 | 3,541,422 | 14.1 | 0.1 | 0.0 |
| Chr7 | 24,649,197 | 20,856,295 | 84.6 | 16,944,007 | 68.7 | 3,497,664 | 14.2 | 0.1 | 0.0 |
| Chr8 | 22,951,293 | 18,276,074 | 79.6 | 14,348,243 | 62.5 | 3,149,084 | 13.7 | 0.1 | 0.0 |
| Mitochondria | 366,924 | 261,869 | 71.4 | 240,190 | 65.5 | 231,177 | 63.0 | 22.5 | 0.0 |
| Chloroplast | 154,478 | 138,839 | 89.9 | 117,414 | 76.0 | 113,021 | 73.2 | 50.3 | 7.8 |

TABLE 4
The percentage of waste reads because of organelle genome
contamination in non-depleted libraries and organelle genome depleted libraries.

| | | | | | |
|---|---|---|---|---|---|
| | | Total | Mapped to | Mapped to | Percentage |
| Sample | Reads | reads | Chloroplast | Mitochondria | organelle genome |

Non-depleted libraries (normal whole genome DNA libraries)

| | | | | | |
|---|---|---|---|---|---|
| A. lyrata-S1 | 100 bp Single-End | 50,834,934 | 13,227,688 | 1,285,640 | 28.55% |
| A. lyrata-S2 | 100 bp Paired-End | 45,245,323 | 12,604,441 | 2,771,797 | 33.98% |

Organelle genome depleted libraries (this invention)

| | | | | | |
|---|---|---|---|---|---|
| A. lyrata-S1 | 150 bp Paired-End | 84,040,304 | 2,926,884 | 1,285,540 | 5.01% |
| A. lyrata-S2 | 150 bp Paired-End | 138,203,286 | 5,217,794 | 2,160,791 | 5.34% |

### REFERENCES

Allum, F, Shao, X, Guénard, F, Simon, M-M, Busche, S, Caron, M, Lambourne, J, Lessard, J, Tandre, K, Hedman, Å K, Kwan, T, Ge, B, Rönnblom, L, McCarthy, M I, Deloukas, P, Richmond, T, Burgess, D, Spector, T D, Tchernof, A, Marceau, S, Lathrop, M, Vohl, M-C, Pastinen, T, Grundberg, E (2015) Characterization of functional methylomes by next-generation capture sequencing identifies novel disease-associated variants. Nature Communications 6, 7211.
Chenchik, A, Diachenko, L, Moqadam, F, Tarabykin, V, Lukyanov, S, and Siebert, P. D., (1996) Full-length cDNA Cloning and Determination of mRNA 5′ and 3′ Ends by Amplification of Adaptor-Ligated cDNA, BioTechniques 21:526-534.
Gnirke, A, Melnikov, A, Maguire, J, Rogov, P, LeProust, E M, Brockman, W, Fennell, T, Giannoukos, G, Fisher, S, Russ, C, Gabriel, S, Jaffe, D B, Lander, E S, Nusbaum, C (2009) Solution Hybrid Selection with Ultra-long Oligonucleotides for Massively Parallel Targeted Sequencing. Nature biotechnology 27, 182-189.
Kato, N, Reynolds, D, Brown, M L, Boisdore, M, Fujikawa, Y, Morales, A, Meisel, L A (2008) Multidimensional fluorescence microscopy of multiple organelles in Arabidopsis seedlings. Plant Methods 4, 9.
Lee, E-J, Pei, L, Srivastava, G, Joshi, T, Kushwaha, G, Choi, J-H, Robertson, K D, Wang, X, Colbourne, J K, Zhang, L, Schroth, G P, Xu, D, Zhang, K, Shi, H (2011) Targeted bisulfite sequencing by solution hybrid selection and massively parallel sequencing. Nucleic Acids Research 39, e127-e127.
Li, Q, Suzuki, M, Wendt, J, Patterson, N, Eichten, S R, Hermanson, P J, Green, D, Jeddeloh, J, Richmond, T, Rosenbaum, H, Burgess, D, Springer, N M, Greally, J M (2015) Post-conversion targeted capture of modified cytosines in mammalian and plant genomes. Nucleic Acids Research 43, e81-e81.
Lister, R, O'Malley, R C, Tonti-Filippini, J, Gregory, B D, Berry, C C, Millar, A H, Ecker, J R (2008) Highly Integrated Single-Base Resolution Maps of the Epigenome in Arabidopsis. Cell 133, 523-536.
Lutz, K A, Wang, W, Zdepski, A, Michael, T P (2011) Isolation and analysis of high quality nuclear DNA with reduced organellar DNA for plant genome sequencing and resequencing. BMC Biotechnology 11, 54.
Ossowski, S, Schneeberger, K, Clark, R M, Lanz, C, Warthmann, N, Weigel, D (2008) Sequencing of natural strains of Arabidopsis thaliana with short reads. Genome Research 18, 2024-2033.
Quispe-Tintaya, W, White, R R, Popov, V N, Vijg, J, Maslov, A Y (2013) Fast mitochondrial DNA isolation from mammalian cells for next-generation sequencing. Bio Techniques 55, 133-136.
Rauwolf, U, Golczyk, H, Greiner, S, Herrmann, RG (2010) Variable amounts of DNA related to the size of chloroplasts III. Biochemical determinations of DNA amounts per organelle. Molecular Genetics and Genomics 283, 35-47.
Shaver, J M, Oldenburg, D J, Bendich, A J (2006) Changes in chloroplast DNA during development in tobacco, Medicago truncatula, pea, and maize. Planta 224, 72-82.
Urich, M A, Nery, J R, Lister, R, Schmitz, R J, Ecker, J R (2015) MethylC-seq library preparation for base-resolution whole-genome bisulfite sequencing. Nat. Protocols 10, 475-483.
Warr, A, Robert, C, Hume, D, Archibald, A, Deeb, N, Watson, M (2015) Exome Sequencing: Current and Future Perspectives. G3: Genes/Genomes/Genetics 5, 1543-1550.
Ziller, M J, Stamenova, E K, Gu, H, Gnirke, A, Meissner, A (2016) Targeted bisulfite sequencing of the dynamic DNA methylome. Epigenetics & Chromatin 9, 55.
Zoschke, R, Liere, K, Börner, T (2007) From seedling to mature plant: Arabidopsis plastidial genome copy number, RNA accumulation and transcription are differentially regulated during leaf development. The Plant Journal 50, 710-722.

1. A method for preparing RNA probes for exome sequencing and/or exome-bisulfite sequencing, the method comprising the steps of:
a) extracting and isolating total RNA from a cell or tissue sample of a eukaryote of interest or providing a ready-made total eukaryotic RNA sample;
b) separating mRNA from the total RNA to obtain a portion of enriched population of mRNA molecules and a portion of non-protein coding RNA;
c) preparing a first adaptor ligated cDNA library from said portion of enriched population of mRNA molecules;
d) preparing a second adaptor ligated cDNA library from said portion of non-protein coding RNA;
e) performing PCR enrichment of said second adaptor ligated cDNA library with a first primer pair comprising an RNA polymerase promoter sequence, wherein said first primer pair also comprises a sequence specific to the adaptor sequence present in the second adaptor ligated cDNA library;
f) synthesizing a first set of RNA probes by using an RNA polymerase in the presence of the enriched cDNA library obtained from step e), wherein said RNA probes are synthesized with a selectable label;
g) hybridizing said first set of RNA probes with said first adaptor ligated cDNA library, separating the hybridized and non-hybridized sample and collecting the non-hybridized sample to produce a depleted-mRNA-library;
h) performing PCR enrichment of said depleted-mRNA-library with a second primer pair comprising an RNA polymerase promoter sequence, wherein said second primer pair also comprises a sequence specific to the adaptor sequence present in the first adaptor ligated cDNA library; and
i) synthesizing a second set of RNA probes suitable for exome sequencing and/or exome-bisulfite sequencing by using an RNA polymerase in the presence of the enriched depleted-mRNA-library obtained from step h), wherein said second set of RNA probes are synthesized with a selectable label.

2. The method according to claim 1, wherein an aliquot of said portion of enriched population of mRNA molecules obtained in step b) is used for normalization in step d).

3. The method according to claim 1, wherein in step c) after adaptor ligation or in step h) after PCR enrichment, a duplex-specific nuclease (DSN) is used to normalize the cDNA library obtained.

4. The method according to claim 1, wherein said RNA probes synthesized in step f) and i) comprise a selectable affinity label.

5. The method according to claim 4, wherein said selectable affinity label is biotin or a derivative thereof.

6. The method according to claim 1, further comprising capturing exome sequences from a DNA library by contacting the second set of RNA probes obtained in step i) with said library and selecting those sequences from said library which are bound to any of said RNA probes.

7. The method according to claim 6, further comprising sequencing the sequences bound to any of said RNA probes.

8. The method according to claim 7, wherein said sequencing is performed as bisulfite sequencing.

9. The method according to claim 1, wherein said RNA polymerase is a SP6, T3 or T7 phage RNA polymerase.

10. A method for preparing RNA capturing probes for the separation of circular DNA from nuclear genome, the method comprising the steps of:
a) extracting and isolating total DNA from a cell or tissue sample of a eukaryote of interest or providing a ready-made total eukaryotic DNA sample;
b) digesting linear nuclear DNA obtained in step a) in the presence of exonucleases or separating circular DNA from the total DNA to isolate organelle DNA and other circular DNA, or providing a ready-made sample of isolated organelle DNA;
c) fragmenting the circular DNA obtained in step b);
d) performing end repairing and dA-tailing to fragments obtained in step c);
e) performing adaptor ligation to fragments obtained in step d) to produce a DNA library of circular DNA fragments;
f) performing PCR enrichment to the DNA library obtained in step e) with a primer pair comprising an RNA polymerase promoter sequence, wherein said primer pair comprises a sequence specific to the adaptor sequence present in the DNA library; and
g) synthesizing a set of RNA probes by using an RNA polymerase in the presence of the enriched DNA library obtained from step f), wherein said set of RNA probes are suitable for depletion of fragmented circular DNA from DNA libraries of said eukaryote of interest and wherein said RNA probes are synthesized with a selectable label.

11. The method according to claim 10, wherein said RNA probes synthesized in step g) comprise a selectable affinity label.

12. The method according to claim 11, wherein said selectable affinity label is biotin or a derivative thereof.

13. The method according to claim 10, wherein said circular DNA is organelle DNA or transposable element DNA.

14. The method according to claim 13, wherein said organelle is chloroplast or mitochondrion.

15. (canceled)

16. The method according to claim 10, further comprising capturing fragmented circular DNA from a DNA library by contacting the set of RNA probes obtained in step g) with said library and separating those sequences from said library which are bound to any of said RNA probes from those sequences which are not bound to any of said RNA probes.

17. The method according to claim 16, further comprising a sequencing the sequences bound to the RNA probes or alternatively the sequences not bound to the RNA probes.

18. The method according to claim 10, wherein said RNA polymerase is a SP6, T3 or T7 phage RNA polymerase.

19-29. (canceled)

30. A kit for exome probe preparation or organelle depletion probe preparation comprising a first and a second adaptor oligonucleotide for cDNA or DNA library preparation, wherein said adaptor oligonucleotides are at least partly complementary to each other, and a primer pair for PCR enrichment, wherein the first primer of said primer pair has a 3′ end specific or complementary to the first adaptor oligonucleotide and a 5′ tail comprising a RNA polymerase promoter sequence and the second primer comprises a sequence which is specific or complementary to the second adaptor oligonucleotide.

31. The kit according to claim 30, wherein said second adaptor oligonucleotide and the second primer have identical sequences.

32. The kit according to claim 30, wherein the length of the first and second adaptor oligonucleotides is 18-25 nt.
